(19) United States
(12) Patent Application Publication
(10) Pub. No.: US 2017/0252537 A1
(43) Pub. Date: Sep. 7, 2017

(54) ENDOSCOPE SHEATH AND ENDOSCOPE INJECTION POSITIONING DEVICE

(71) Applicant: OLYMPUS CORPORATION, Tokyo (JP)

(72) Inventors: Masaki HAYASHI, Tokyo (JP); Takahiro ONO, Tokyo (JP)

(73) Assignee: OLYMPUS CORPORATION, Tokyo (JP)

(21) Appl. No.: 15/600,102

(22) Filed: May 19, 2017

Related U.S. Application Data
(63) Continuation of application No. PCT/JP2014/081265, filed on Nov. 26, 2014.

(51) Int. Cl. A61M 25/00; A61M 25/06; A61B 1/018; A61M 5/32

(52) U.S. Cl. CPC A61M 25/0084; A61M 5/3287; A61M 25/06; A61B 1/018; A61M 2025/0089; A61M 2005/3289; A61B 2017/0034

(57) ABSTRACT

Provided is an endoscope sheath including: a sheath body having an elongated rotating section and a stationary section, which are coupled to each other in a relatively rotatable manner about a longitudinal axis; a first passage along the axis; a second passage parallel to the first passage; an outlet through which a proximal end of an injection needle inserted in the second passage is pulled outside, the outlet being capable of changing a pullout position of the proximal end of the needle relative to the stationary section in the circumferential direction; and holders provided in the stationary section and separated by intervals in the circumferential direction, each holder securing an injector having the needle to the stationary section at a position where the needle protrudes from the distal end of the body and releasing the injector at a position where the needle is accommodated inside the body.

### CROSS-REFERENCE TO RELATED APPLICATIONS

This is a continuation of International Application PCT/JP2014/081265 which is hereby incorporated by reference herein in its entirety.

### TECHNICAL FIELD

The present invention relates to endoscope sheaths and endoscope injection positioning devices.

### BACKGROUND ART

A trans-endoscopic method used as a method of treating stress urinary incontinence in the related art involves inserting an endoscope into the urethra and injecting a gel-like drug solution, such as a collagen solution, into the urethral wall by using an injection needle so as to cause the urethral wall to locally bulge (for example, see Patent Literature 1). The injection of the drug solution is normally performed at three locations separated by intervals of 120° in the circumferential direction so that the urethral wall bulges evenly over the entire circumference.

Patent Literature 1 discloses an endoscope sheath as a device for assisting with the injection process performed at three locations. Specifically, the endoscope sheath is attached to the outer side of the endoscope in such a manner that the endoscope sheath is rotatable about the longitudinal axis relative to the endoscope. A passage for the injection needle extends in the longitudinal direction through the sidewall of the sheath. By rotating the sheath relative to the endoscope, the piercing position of the injection needle protruding from the distal end of the sheath via the passage is rotated in the circumferential direction of the endoscope, so that the three injection processes can be performed sequentially.

### CITATION LIST

### Patent Literature

{PTL 1}
Publication of Japanese Patent No. 4616253

### SUMMARY OF INVENTION

A first aspect of the present invention provides an endoscope sheath including an elongated sheath body, a first passage, a second passage, an outlet, and a plurality of holders. The sheath body has an elongated rotating section at a distal side thereof and a stationary section at a proximal side thereof. The rotating section and the stationary section are coupled to each other in a relatively rotatable manner about a longitudinal axis. The first passage extends through the sheath body along the longitudinal axis from a distal-end surface to a proximal-end surface thereof. An insertion section of an endoscope is insertable into the first passage. The second passage is formed, in the sheath body, parallel to the first passage from the distal-end surface toward the proximal side. An injection needle is insertable into the second passage. A proximal end of the injection needle inserted in the second passage is pulled outside the sheath body through the outlet. The outlet communicates with a proximal end of the second passage and is provided at a radially outer side of the sheath body. The plurality of holders are provided in the stationary section and are separated by intervals in a circumferential direction around the longitudinal axis. The holders hold an injector having the injection needle pulled out through the outlet. Each holder secures the injector, which is disposed at a protruding position where a distal end of the injection needle protrudes from a distal end of the sheath body via the second passage, to the stationary section in the circumferential direction and releases the injector at a released position located toward the proximal side relative to the protruding position. The released position is where the distal end of the injection needle is accommodated inside the sheath body. In a state where the injector is released from the holder, the outlet is capable of changing a pullout position of the proximal end of the injection needle relative to the stationary section in the circumferential direction as the rotating section and the stationary section are relatively rotated.

A second aspect of the present invention provides an endoscope injection positioning device which is used together with an endoscope sheath having a first passage that extends therethrough along a longitudinal axis from a distal-end surface to a proximal-end surface thereof and into which an insertion section of an endoscope is insertable, and also having a second passage that is formed parallel to the first passage from the distal-end surface to the proximal-end surface and into which an injection needle is insertable. The endoscope injection positioning device includes a substantially-cylindrical body having a passage into which the insertion section is insertable. The body includes a plurality of engagement holes, a circular-arc-shaped or ring-shaped guide groove, a peripheral groove, and a plurality of vertical grooves. The plurality of engagement holes are separated by intervals in a circumferential direction in the proximal-end surface. Each engagement hole is engageable, in a central-axis direction from a proximal side, with at least a distal end of an injector connected to a proximal end of the injection needle inserted in the second passage. The plurality of engagement holes alternately connect with the second passage in the central-axis direction in accordance with a relative angle with the endoscope sheath around the longitudinal axis. The circular-arc-shaped or ring-shaped guide groove connects ends of the plurality of engagement holes at a distal side. The peripheral groove extends in the circumferential direction in an outer peripheral surface. The plurality of vertical grooves are formed in the outer peripheral surface and extend from the proximal-end surface to the peripheral groove so as to connect the engagement holes and the peripheral groove. The guide groove, the peripheral groove, and the plurality of vertical grooves each have a width larger than a diameter of the injection needle, and the peripheral groove and the plurality of vertical grooves each have a width smaller than a diameter of the injector.

### BRIEF DESCRIPTION OF DRAWINGS

FIG. 1 illustrates the overall configuration of an endoscope injection kit provided with an endoscope sheath according to a first embodiment of the present invention.

FIG. 2 is a cross-sectional view taken along line I-I, illustrating the configuration of a rotating section in the endoscope sheath in FIG. 1.

FIG. 3 is a vertical sectional view illustrating the shape of a second passage in a distal-end area of a sheath body.

FIG. 4 is a cross-sectional view taken along line II-II, illustrating the configuration of a stationary section in the endoscope sheath in FIG. 1.

FIG. 5 illustrates a modification of an injector used in the endoscope sheath in FIG. 1.

FIG. 6 illustrates the overall configuration of an endoscope injection kit provided with an endoscope sheath according to a second embodiment of the present invention.

FIG. 7 is a six-sided view illustrating the configuration of a stationary section in the endoscope sheath in FIG. 6.

FIG. 8 is a perspective view of the stationary section in FIG. 7 and illustrates a method for moving an injection needle.

### DESCRIPTION OF EMBODIMENTS

### First Embodiment

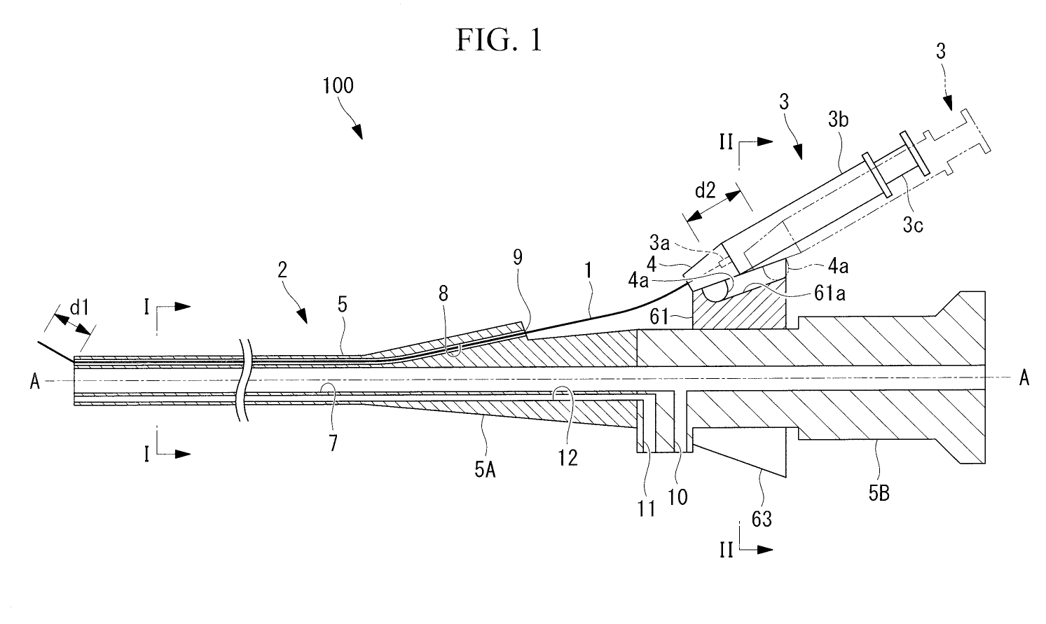

An endoscope injection kit 100 according to a first embodiment of the present invention will be described below with reference to FIGS. 1 to 5.

As shown in FIG. 1, the injection kit 100 according to this embodiment is used together with a urethral endoscope and includes a thin, hollow injection needle (injector) 1, connected to a syringe (injector) 3, and an endoscope sheath 2.

The endoscope includes an elongated insertion section insertable into the urethra and an operable section connected to the proximal end of the insertion section.

The syringe 3 includes a tubular cylinder 3b that accommodates a drug solution therein and a piston 3c inserted in the cylinder 3b. By pressing the piston 3c, the drug solution in the cylinder 3b can be supplied into the injection needle 1 via a discharge port 3a. The drug solution forms a gel at body temperature while flowing smoothly inside the injection needle 1, and is, for example, a collagen solution.

The proximal end of the injection needle 1 is provided with a cap 4. In a state where the proximal end of the injection needle 1 is connected to the discharge port 3a of the syringe 3, the syringe 3 and the cap 4 disposed at the distal end of the syringe 3 are secured together. The cap 4 has a substantially-cylindrical outer peripheral surface provided with a protrusion 4a protruding in the radial direction.

The endoscope sheath 2 includes an elongated sheath body 5 attached to the outer side of the insertion section of the endoscope and three holders 61, 62, and 63 provided at the proximal-end area of the sheath body 5 for holding the syringe 3.

The sheath body 5 is divided into two sections in the longitudinal direction, which are a rotating section 5A at the distal side and a stationary section 5B at the proximal side. The rotating section 5A has an elongated cylindrical shape capable of accommodating substantially the entire insertion section lengthwise. The rotating section 5A and the stationary section 5B are coaxially coupled to each other in a relatively rotatable manner about a longitudinal axis A.

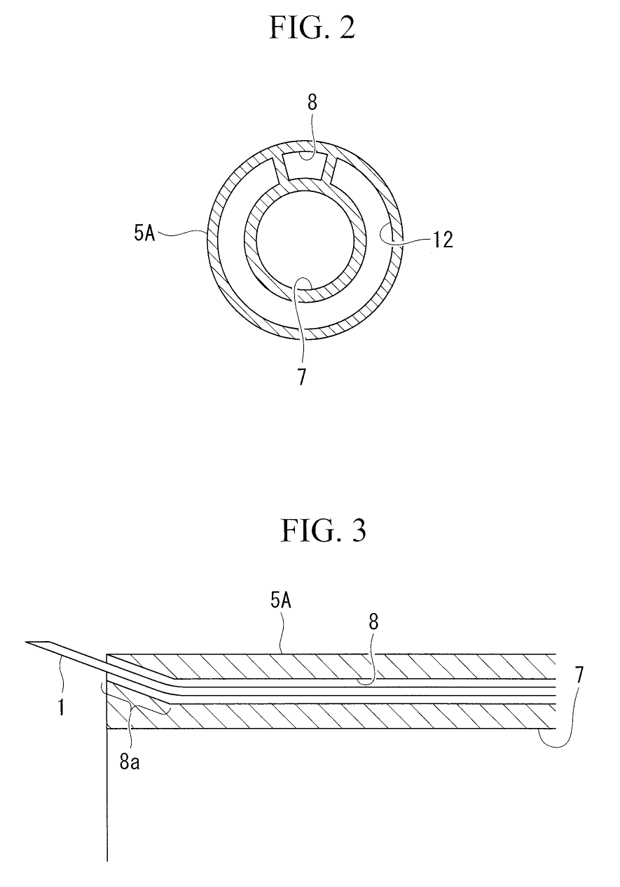

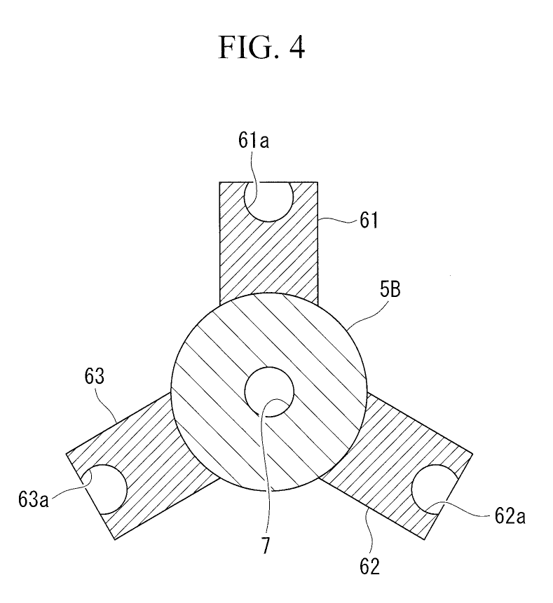

FIG. 2 is a cross-sectional view of the rotating section 5A, and FIG. 4 is a cross-sectional view of the stationary section 5B. As shown in FIGS. 1, 2, and 4, the sheath body 5 has a first passage 7 and a second passage 8 that extend in the longitudinal direction and that are formed parallel to each other.

The first passage 7 is a passage for the insertion section of the endoscope and is a cylindrical space extending through the sheath body 5 along the longitudinal axis A thereof from the distal-end surface to the proximal-end surface of the sheath body 5. The first passage 7 has an inner diameter slightly larger than the outer diameter of the insertion section. The sheath body 5 and the insertion section inserted into the first passage 7 from the proximal side are relatively movable in the longitudinal direction and are also relatively rotatable about the longitudinal axis A.

The second passage 8 is a passage for the injection needle 1 and extends in the longitudinal direction at the radially outer side of the first passage 7 from the distal-end surface toward the proximal side of the sheath body 5. The proximal-end area of the rotating section 5A has a needle feed port (outlet) 9 at the radially outer side, and the proximal end of the second passage 8 is connected to the needle feed port 9. The second passage 8 has an inner diameter larger than the outer diameter of the injection needle 1. The rotating section 5A and the injection needle 1 inserted into the second passage 8 via the needle feed port 9 are relatively movable in the longitudinal direction. As shown in FIG. 3, the distal-end area of the second passage 8 constitutes an inclined area 8a that is inclined relative to the longitudinal axis A gradually away from the longitudinal axis A toward the distal end of the rotating section 5A. An angle formed between the inclined area 8a and the longitudinal axis A is an angle suitable for piercing the urethral wall with the injection needle 1 and is specifically set between 30° and 40°.

Furthermore, the sheath body 5 has a feed port 10 and a drain port 11 provided in the stationary section 5B and also has a liquid channel 12. The feed port 10 communicates with the first passage 7. A liquid (e.g., lavage fluid) injected into the first passage 7 via the feed port 10 passes through a gap between the inner peripheral surface of the first passage 7 and the outer peripheral surface of the insertion section and is discharged from an opening in the distal-end surface of the sheath body 5. The liquid channel 12 extends through the sheath body 5 from the distal-end surface thereof to the drain port 11. The interior of the liquid channel 12 is suctioned from the drain port 11 so that the liquid near the distal-end surface of the sheath body 5 is suctioned into the liquid channel 12 and is drained from the drain port 11.

As shown in FIG. 4, the holders 61, 62, and 63 are provided on the outer peripheral surface of the stationary section 5B at three locations separated by equal intervals in the circumferential direction. The holders 61, 62, and 63 are block-like members and respectively have engagement holes 61a, 62a, and 63a at the radially outer side and the proximal side of the sheath body 5. The engagement holes 61a, 62a, and 63a extend substantially in the longitudinal direction from the proximal side toward the distal side of the sheath body 5 and end at intermediate positions between the proximal ends and the distal ends of the holders 61, 62, and 63.

Each of the engagement holes 61a, 62a, and 63a has a shape such that it is engageable with the protrusion 4a of the cap 4 substantially in the longitudinal direction of the sheath body 5 from the proximal side thereof. The cap 4, with its protrusion 4a engaged with the engagement hole 61a, 62a, or 63a, and the syringe 3 secured to the cap 4 are secured to the stationary section 5B in the circumferential direction around the longitudinal axis A and are restricted from moving any further toward the distal side at a maximum protruding position (protruding position) where the protrusion 4a abuts on the inner wall of the terminal end of the engagement hole 61a, 62a, or 63a.

The overall length of the injection needle 1 is designed such that, when the syringe 3 is disposed at the maximum protruding position (see the solid line in FIG. 1), the distal end of the injection needle 1 protrudes by a predetermined length d1 from the distal-end surface of the sheath body 5. Furthermore, the lengthwise dimension of each of the engagement holes 61a, 62a, and 63a is designed such that a moving distance d2 of the syringe 3 between the maximum protruding position and a recessed position (see the two-dot chain line in FIG. 1) where the protrusion 4a is positioned at the proximal end of the engagement hole 61a, 62a, or 63a is larger than the length d1.

Next, the operation of the injection kit 100 having the above-described configuration will be described with reference to an example in which stress urinary incontinence is treated.

In order to treat stress urinary incontinence by using the injection kit 100 according to this embodiment, the insertion section of the endoscope is first inserted into the first passage 7 of the sheath body 5 from the proximal side thereof so as to attach the sheath body 5 to the insertion section. Then, the insertion section with the sheath body 5 attached thereto is inserted into the urethra, and the distal end of the insertion section is positioned at an appropriate position within the urethra. Subsequently, the rotating section 5A is rotated while the insertion section and the stationary section 5B are kept fixed in position, so that the orientation (i.e., rotational angle) of the needle feed port 9 around the longitudinal axis A is substantially aligned with one holder 61.

Then, the injection needle 1 is inserted into the second passage 8 via the needle feed port 9, and the protrusion 4a of the cap 4 provided at the proximal end of the injection needle 1 extending outward from the sheath body 5 via the needle feed port 9 is engaged with the engagement hole 61a, whereby the syringe 3 is held by the holder 61 by means of the cap 4. By sliding the syringe 3 along the engagement hole 61a toward the distal side until it reaches the maximum protruding position, the distal end of the injection needle 1 can be made to protrude by the predetermined distance d1 from the distal-end surface of the sheath body 5. The distal end of the injection needle 1 can be observed in an endoscopic image.

The inclined area 8a provided in the distal-end area of the second passage 8 causes the distal end of the injection needle 1 to protrude diagonally forward toward the radially outer side of the sheath body 5 at a tilt angle of 30° to 40° relative to the longitudinal axis A. Therefore, the urethral wall laterally adjoining the sheath body 5 can be pierced with the injection needle 1 at an appropriate angle between 30° and 40°.

Subsequently, the drug solution in the syringe 3 is injected into the urethral wall from the distal end of the injection needle 1. Consequently, the drug solution injected into the urethral wall causes the urethral wall to locally bulge.

Then, the syringe 3 is moved toward the proximal side until it reaches a position (released position) where the protrusion 4a is disposed further toward the proximal side relative to the proximal end of the engagement hole 61a, so that the syringe 3 can be removed from the holder 61. Consequently, the syringe 3 is released from the holder 61 and becomes movable both in the circumferential direction and the radial direction relative to the stationary section 5B. In this case, since the moving distance d2 over which the syringe 3 moves toward the proximal side, which is required for removing the syringe 3 from the holder 61, is larger than the length d1 of the distal end of the injection needle 1 protruding from the distal end of the sheath body 5, the needle tip of the injection needle 1 reliably retracts into the sheath body 5 further inward than the distal end thereof as the syringe 3 is removed from the holder 61.

Subsequently, the rotating section 5A is rotated by 120° while the insertion section and the stationary section 5B are kept fixed in position, so that the orientation of the needle feed port 9 around the longitudinal axis A is substantially aligned with another holder 62. Then, the syringe 3 is held by the holder 62, and a second injection process is performed on the urethral wall in a manner similar to the first injection process.

Subsequently, the syringe 3 is removed from the holder 62, and the rotating section 5A is rotated by 120° while the insertion section and the stationary section 5B are kept fixed in position, so that the orientation of the needle feed port 9 around the longitudinal axis A is substantially aligned with the remaining holder 63. Then, the syringe 3 is held by the holder 63, and a third injection process is performed on the urethral wall in a manner similar to the first injection process.

Accordingly, the drug solution can be injected into the urethral wall at three locations separated by angular intervals of 120° in the circumferential direction.

In this case, in this embodiment, the syringe 3 is held by the holder 61, 62, or 63 between the maximum protruding position, at which the needle tip protrudes from the distal-end surface of the sheath body 5, and the recessed position so that the syringe 3 does not move in the circumferential direction relative to the stationary section 5B. Moreover, when the syringe 3 is released from the holder 61, 62, or 63, the needle tip is reliably accommodated inside the sheath body 5. This is advantageous in that the injection needle 1 is prevented from rotating within the urethra in a state where the injection needle 1 is exposed from the sheath body 5, and that the injection needle 1 can be reliably prevented from coming into contact with an area other than the injection position of the urethral wall.

Furthermore, since the distal end of the syringe 3 is positionally limited by the terminal end of the engagement hole 61a, 62a, or 63a, the length of the injection needle 1 protruding from the distal-end surface of the sheath body 5 is limited to within the predetermined length d1. This is advantageous in that the depth to which the urethral wall is pierced with the injection needle 1 can be limited. Moreover, each injection position is determined in accordance with the circumferential position, around the longitudinal axis A, of the holder 61, 62, or 63 holding the syringe 3. This is advantageous in that, by performing three injection processes while sequentially causing the three holders 61, 62, and 63 to hold the syringe 3, the three injection positions can be properly and readily set to the three locations separated by equal intervals in the circumferential direction in correspondence with the three holders 61, 62, and 63 arranged in the stationary section 5B.

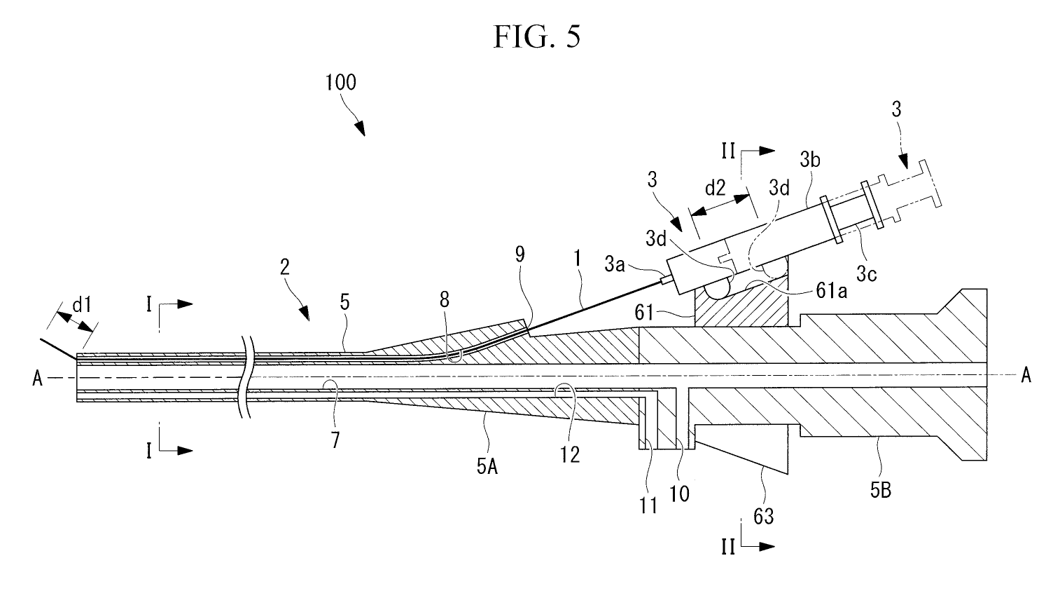

In this embodiment, the holder 61, 62, or 63 holds the syringe 3 by means of the cap 4 integrally secured to the syringe 3. Alternatively, as shown in FIG. 5, a protrusion 3d provided on the outer peripheral surface of the cylinder 3b may be engaged with the engagement hole 61a, 62a, or 63a so that the holder 61, 62, or 63 directly holds the syringe 3. The protrusion 3d may be integrated with the cylinder 3b or may be a component that is attachable to or detachable from the cylinder 3b.

### Second Embodiment

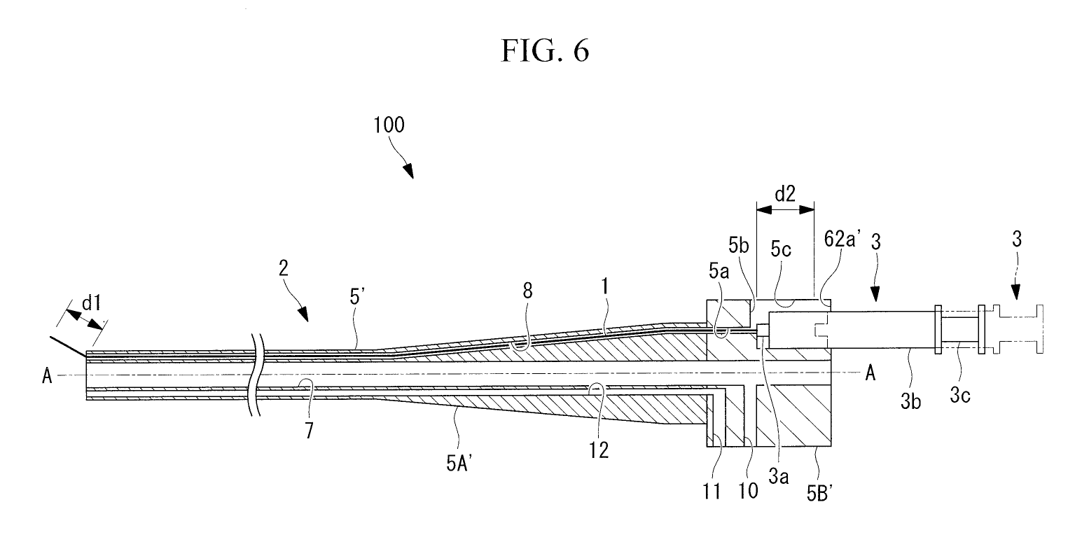

Next, an endoscope injection kit 100 according to a second embodiment of the present invention will be described with reference to FIGS. 6 to 8.

As shown in FIG. 6, the injection kit 100 according to this embodiment mainly differs from that in the first embodiment in terms of the configuration of a stationary section 5B′ of the endoscope sheath 2. Therefore, in this embodiment, the stationary section 5B′ will be mainly described, and components identical to those in the first embodiment will be given the same reference signs and will not be described.

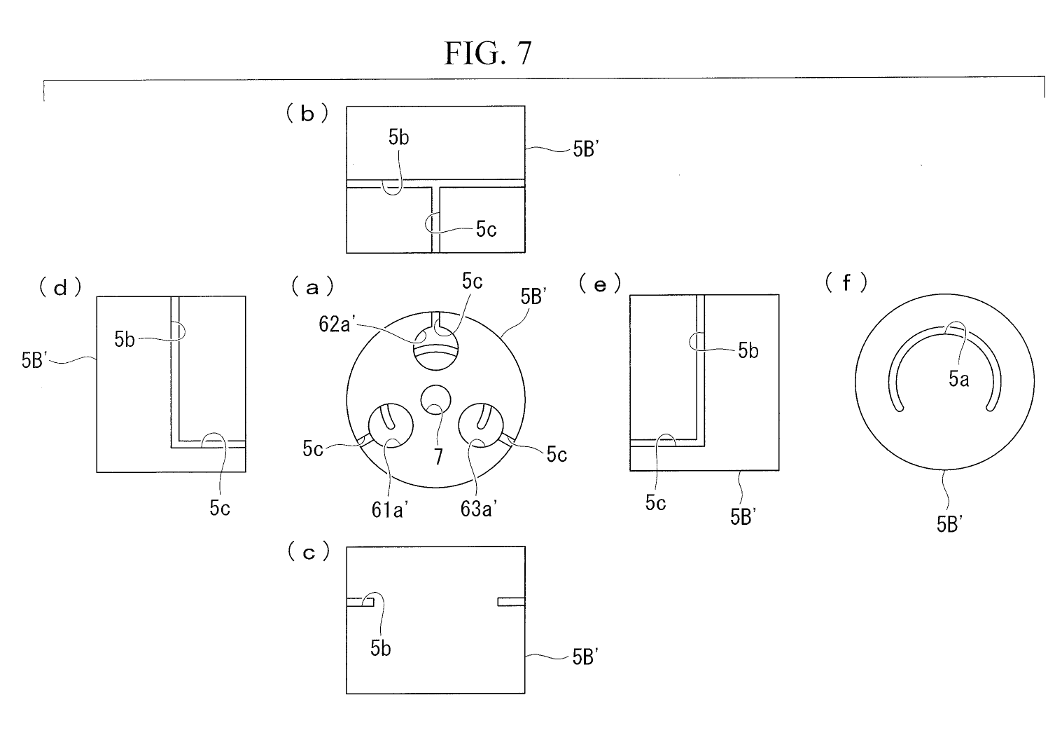

FIG. 7 includes a front view (a) of the stationary section 5B′, as viewed from the proximal side, a plan view (b), a bottom view (c), a left side view (d), a right side view (e), and a rear view (f). In FIG. 7, the feed port 10 and the drain port 11 are not shown.

As shown in FIG. 7, the stationary section 5B′ is a cylindrical member and has, on the surface thereof, three engagement holes 61a′, 62a′, and 63a′, a guide groove 5a, a peripheral groove 5b, and three vertical grooves 5c.

The engagement holes 61a′, 62a′, and 63a′ are formed in the proximal-end surface of the stationary section 5B′ at three locations separated by equal intervals in the circumferential direction and are engageable with the distal end of the syringe 3 from the proximal side. The engagement holes 61a′, 62a′, and 63a′ end at intermediate positions between the proximal-end surface and the distal-end surface of the stationary section 5B′. When the syringe 3 is disposed at a maximum protruding position where the distal end of the syringe 3 abuts on the terminal end of the engagement hole 61a′, 62a′, or 63a′ (see the solid line in FIG. 6), the distal end of the injection needle 1 protrudes by a predetermined distance d1 from the distal-end surface of the sheath body 5′. Furthermore, the lengthwise dimension of each of the engagement holes 61a′, 62a′, and 63a′ is designed such that a moving distance d2 of the syringe 3 between the maximum protruding position and a recessed position (see the two-dot chain line in FIG. 6) where the distal end of the syringe 3 is positioned at the proximal end of the engagement hole 61a′, 62a′, or 63a′ is larger than the length d1.

The guide groove 5a is formed in a circular-arc shape or ring shape centered on the longitudinal axis A in the distal-end surface of the stationary section 5B′ so as to connect, in the circumferential direction, the ends of the three engagement holes 61a′, 62a′, and 63a′ at the distal side. In the rotating section 5A′ according to this embodiment, the needle feed port 9 is eliminated, and the second passage 8 extends through the rotating section 5A′ in the longitudinal direction from the distal-end surface to the proximal-end surface. The radius of the guide groove 5a centered on the longitudinal axis A is substantially equal to the distance, in the radial direction, from the longitudinal axis A to the second passage 8 in the proximal-end surface of the rotating section 5A′. Thus, in accordance with the relative angle between the rotating section 5A′ and the stationary section 5B′ around the longitudinal axis A, the three engagement holes 61a′, 62a′, and 63a′ alternately connect with the second passage 8 of the rotating section 5A′ via the guide groove 5a in the longitudinal direction.

The peripheral groove 5b is formed in the circumferential direction in the outer peripheral surface of the stationary section 5B′ so as to extend from a position adjacent to one engagement hole 61a′ in the radial direction to a position adjacent to another engagement hole 63a′ in the radial direction via a position adjacent to the remaining engagement hole 62a′ in the radial direction.

The vertical grooves 5c are formed in the longitudinal direction in the outer peripheral surface of the stationary section 5B′ from the proximal-end surface of the stationary section 5B′ to the peripheral groove 5b so as to connect the respective engagement holes 61a′, 62a′, and 63a′ to the peripheral groove 5b.

The widths of the guide groove 5a, the peripheral groove 5b, and the vertical grooves 5c are designed to be larger than the outer diameter of the injection needle 1. Furthermore, the widths of the peripheral groove 5b and the vertical grooves 5c are designed to be smaller than the outer diameter of the syringe 3. Thus, the injection needle 1 is capable of moving within the grooves 5a, 5c, and 5c, and the syringe 3 is not allowed to be inserted into the peripheral groove 5b and the vertical grooves 5c.

Next, the operation of the injection kit 100 having the above-described configuration will be described.

The endoscope sheath 2 according to this embodiment is used differently from that in the first embodiment in terms of how the syringe 3 is attached to the stationary section 5B′ and how the syringe 3 is moved among the engagement holes 61a′, 62a′, and 63a′.

In order to treat stress urinary incontinence by using the injection kit 100 according to this embodiment, as in the first embodiment, the insertion section with the sheath body 5′ attached thereto is inserted into the urethra and is positioned therein, and the rotating section 5A′ is rotated while the insertion section and the stationary section 5B′ are kept fixed in position, so that the orientation of the second passage 8 around the longitudinal axis A is substantially aligned with one engagement hole 61a′.

Then, the injection needle 1 is inserted into the second passage 8 via the engagement hole 61a′ and the guide groove 5a, and the distal end of the syringe 3 connected to the proximal end of the injection needle 1 extending from the engagement hole 61a′ to the outer side of the sheath body 5′ is engaged with the engagement hole 61a′, whereby the syringe 3 is held by the engagement hole 61a′. By sliding the syringe 3 within the engagement hole 61a′ toward the distal side until it reaches the maximum protruding position, the distal end of the injection needle 1 can be made to protrude by the predetermined distance d1 from the distal-end surface of the sheath body 5′, so that the urethral wall can be pierced with the injection needle 1. Then, the drug solution is injected into the urethral wall.

In order to move the syringe 3 into another engagement hole 62a′ after the first injection process, the syringe 3 is first moved toward the proximal side until it reaches a position (released position) where the distal end of the syringe 3 is disposed further toward the proximal side than the proximal end of the engagement hole 61a′, so that the syringe 3 can be removed from the holder 61. In this case, since the moving distance d2 over which the syringe 3 moves toward the proximal side, which is required for removing the syringe 3 from the engagement hole 61a′, is larger than the length d1 of the distal end of the injection needle 1 protruding from the distal end of the sheath body 5′, the needle tip of the injection needle 1 is reliably accommodated within the second passage 8 as the syringe 3 is removed from the engagement hole 61a′.

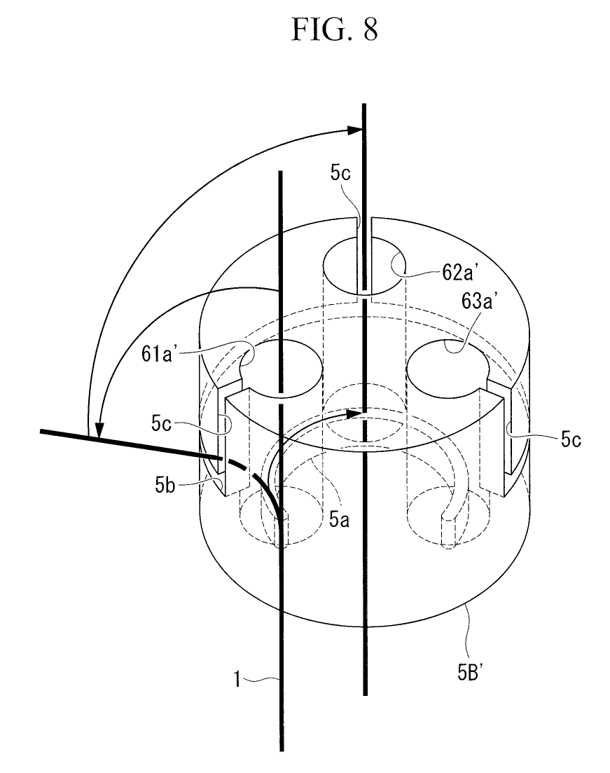

Subsequently, the rotating section 5A′ is rotated by 120° while the insertion section and the stationary section 5B′ are kept fixed in position, so that the orientation of the second passage 8 around the longitudinal axis A is substantially aligned with another engagement hole 62a′. Simultaneously with the rotation of the rotating section 5A′, the injection needle 1 connected to the syringe 3 is moved from the engagement hole 61a′ to the peripheral groove 5b via the corresponding vertical groove 5c, as shown in FIG. 8. Thus, the proximal end of the injection needle 1 is pulled out toward the radially outer side of the sheath body 5′ from the peripheral groove (outlet) 5b, so that the injection needle 1 and the syringe 3 become movable in the circumferential direction relative to the stationary section 5B′ while shifting the pullout position of the injection needle 1 from the peripheral groove 5b in the circumferential direction, whereby the injection needle 1 can be moved to the engagement hole 62a′ from the peripheral groove 5b via another vertical groove 5c. Then, the syringe 3 is held by the engagement hole 62a′, and a second injection process is performed on the urethral wall in a manner similar to the first injection process. In FIG. 8, the first passage 7, the feed port 10, and the drain port 11 are not shown.

Subsequently, the syringe 3 is removed from the engagement hole 62a′, and the rotating section 5A′ is further rotated by 120° while the insertion section and the stationary section 5B′ are kept fixed in position, so that the orientation of the second passage 8 around the longitudinal axis A is substantially aligned with the remaining engagement hole 63a′. Then, the syringe 3 is held by the engagement hole 63a′, and a third injection process is performed on the urethral wall in a manner similar to the first injection process.

In this case, when moving the syringe 3 among the engagement holes 61a′, 62a′, and 63a′ in this embodiment, the position of the syringe 3 is limited to the outer side of the stationary section 5B′ by the narrow peripheral groove 5b and vertical grooves 5c, so that pushing-in of the injection needle 1 toward the distal side is limited. This is advantageous in that the injection needle 1 can be more reliably prevented from rotating within the urethra in a state where the injection needle 1 is exposed from the sheath body 5′. Other advantages of this embodiment are similar to those of the first embodiment.

The stationary section 5B′ described in this embodiment may alone serve as an endoscope injection positioning device. In this case, the feed port 10 and the drain port 11 are not necessary.

The endoscope injection positioning device may be used together with a general-purpose endoscope sheath having a structure similar to the rotating section 5A or 5A′. In a case where the general-purpose endoscope sheath is used by attaching the endoscope injection positioning device to the insertion section at a position further toward the proximal side than the endoscope sheath, advantages similar to those of the first and second embodiments described above can be achieved.

The above-described embodiment leads to the following invention.

A first aspect of the present invention provides an endoscope sheath including an elongated sheath body, a first passage, a second passage, an outlet, and a plurality of holders. The sheath body has an elongated rotating section at a distal side thereof and a stationary section at a proximal side thereof. The rotating section and the stationary section are coupled to each other in a relatively rotatable manner about a longitudinal axis. The first passage extends through the sheath body along the longitudinal axis from a distal-end surface to a proximal-end surface thereof. An insertion section of an endoscope is insertable into the first passage. The second passage is formed, in the sheath body, parallel to the first passage from the distal-end surface toward the proximal side. An injection needle is insertable into the second passage. A proximal end of the injection needle inserted in the second passage is pulled outside the sheath body through the outlet. The outlet communicates with a proximal end of the second passage and is provided at a radially outer side of the sheath body. The plurality of holders are provided in the stationary section and are separated by intervals in a circumferential direction around the longitudinal axis. The holders hold an injector having the injection needle pulled out through the outlet. Each holder secures the injector, which is disposed at a protruding position where a distal end of the injection needle protrudes from a distal end of the sheath body via the second passage, to the stationary section in the circumferential direction and releases the injector at a released position located toward the proximal side relative to the protruding position. The released position is where the distal end of the injection needle is accommodated inside the sheath body. In a state where the injector is released from the holder, the outlet is capable of changing a pullout position of the proximal end of the injection needle relative to the stationary section in the circumferential direction as the rotating section and the stationary section are relatively rotated.

According to the first aspect of the present invention, the sheath body is attached to the outer side of the insertion section of the endoscope by inserting the insertion section into the first passage. Then, the insertion section with the sheath body attached thereto is inserted into the biological body, and the injection needle is inserted into the biological body via the second passage. Thus, a drug solution can be injected by piercing tissue with the injection needle while using the endoscope to observe the needle tip of the injection needle protruding from the distal end of the sheath body. After the first injection process, the needle tip is rotated in the circumferential direction of the endoscope by rotating the rotating section relative to the insertion section while maintaining the position of the insertion section relative to the tissue, so that a subsequent injection process can be performed at another position, separated therefrom by an interval in the circumferential direction of the field of view.

In this case, the first injection process involves determining the orientation of the rotating section relative to the stationary section around the longitudinal axis so that the orientation, around the longitudinal axis (i.e., the rotational angle around the longitudinal axis), of one of the holders provided in the stationary section is substantially aligned with the second passage, and then causing the holder to hold the injector. The second injection process involves determining the orientation of the rotating section relative to the stationary section around the longitudinal axis so that the orientation of another holder around the longitudinal axis is substantially aligned with the second passage, and then causing the holder to hold the injector. Subsequently, the injection is similarly performed multiple times while changing the holders holding the injector. Consequently, the injection can be performed at positions separated by intervals in the circumferential direction in accordance with the circumferential arrangement of the plurality of holders in the stationary section, whereby a plurality of injection positions can be readily and properly determined.

In a state where the injector is released from the holders, the pullout position of the proximal end of the injection needle pulled out to the radially outer side of the sheath body through the outlet is also changeable in the circumferential direction relative to the stationary section as the orientation of the rotating section is changed. Therefore, while the injection needle is kept inserted in the second passage of the sheath body, it is possible to change the holder holding the injector by moving the injector in the released state in the circumferential direction relative to the stationary section.

Furthermore, when the injector is held by the stationary section and is disposed at the protruding position where the needle tip of the injection needle protrudes from the distal end of the sheath body, the injector is secured by one of the holders so as not to move in the circumferential direction of the stationary section. In order to move the injector to another holder, the injector is released from the current holder by being moved from the protruding position to the released position, thus causing the needle tip to retract inside the sheath body. Consequently, the injection needle is prevented from rotating within the biological body while remaining in a state where it protrudes from the sheath body, and the injection needle can be reliably prevented from coming into contact with tissue areas other than the injection position.

In the first aspect described above, a distal-end area of the second passage may be inclined relative to the longitudinal axis gradually away from the longitudinal axis toward the distal end.

Accordingly, the injection needle protrudes from the distal end of the sheath body diagonally forward toward the radially outward side relative to the endoscope, so that a tissue surface located beside the sheath body can be pierced at an angle with the injection needle.

In the first aspect described above, the plurality of holders may have a plurality of engagement holes provided in the stationary section and separated by intervals in the circumferential direction. Each engagement hole may be oriented toward the proximal side and be engageable with at least a part of the injector substantially in a longitudinal direction of the sheath body from the proximal side.

Accordingly, by sliding at least a part of the injector into one of the engagement holes from the proximal side, the injector is held by the engagement hole so as not to move in the circumferential direction relative to the stationary section. Moreover, by sliding the injector toward the proximal side, the injector can be released from the engagement hole.

In the first aspect described above, each engagement hole may hold the injector in a movable manner in the longitudinal direction between a maximum protruding position where further movement of the injector toward the distal side is limited and a recessed position located toward the proximal side relative to the maximum protruding position. The maximum protruding position may be where the injection needle protrudes by a predetermined length from the distal end of the sheath body. A moving distance of the injector between the maximum protruding position and the recessed position may be larger than the predetermined length.

Accordingly, the depth to which the tissue is pierced with the injection needle can be limited, and the injection needle can be reliably recessed inside the sheath body as the injector is released from the engagement hole.

In the first aspect described above, the plurality of engagement holes may be formed in a proximal-end surface of the stationary section and may alternately connect with the second passage in the longitudinal direction in accordance with a relative angle between the rotating section and the stationary section around the longitudinal axis. The stationary section may include a circular-arc-shaped or ring-shaped guide groove that connects ends of the plurality of engagement holes at the distal side, a peripheral groove extending in the circumferential direction in an outer peripheral surface and serving as the outlet, and a plurality of vertical grooves formed in the outer peripheral surface and extending from the proximal-end surface to the peripheral groove so as to connect the engagement holes and the peripheral groove. The guide groove, the peripheral groove, and the plurality of vertical grooves may each have a width larger than a diameter of the injection needle, and the peripheral groove and the plurality of vertical grooves may each have a width smaller than a diameter of the injector.

Accordingly, as the injection needle is inserted into the second passage via one of the engagement holes, the injector can be engaged with the engagement hole. A process for moving the injector to another engagement hole involves releasing the injector from the current engagement hole, moving the proximal end of the injection needle from the engagement hole to the peripheral groove via one of the vertical grooves, moving the proximal end of the injection needle through the peripheral groove to another vertical groove, further moving the proximal end of the injection needle from the vertical groove to another engagement hole, and then engaging the injector with the engagement hole. In this process, the injection needle may be moved through the peripheral groove while rotating the rotating section so that the injection needle can be rotated along the guide groove while being maintained substantially straight. In this case, even in a state where the injector is released from the engagement holes, pushing-in of the injection needle toward the distal side is limited by the vertical grooves and the peripheral groove that are narrower than the injector, so that the injection needle can be reliably prevented from protruding from the distal end of the sheath body while the injector moves from engagement hole to engagement hole.

A second aspect of the present invention provides an endoscope injection positioning device which is used together with an endoscope sheath having a first passage that extends therethrough along a longitudinal axis from a distal-end surface to a proximal-end surface thereof and into which an insertion section of an endoscope is insertable, and also having a second passage that is formed parallel to the first passage from the distal-end surface to the proximal-end surface and into which an injection needle is insertable. The endoscope injection positioning device includes a substantially-cylindrical body having a passage into which the insertion section is insertable. The body includes a plurality of engagement holes, a circular-arc-shaped or ring-shaped guide groove, a peripheral groove, and a plurality of vertical grooves. The plurality of engagement holes are separated by intervals in a circumferential direction in the proximal-end surface. Each engagement hole is engageable, in a central-axis direction from a proximal side, with at least a distal end of an injector connected to a proximal end of the injection needle inserted in the second passage. The plurality of engagement holes alternately connect with the second passage in the central-axis direction in accordance with a relative angle with the endoscope sheath around the longitudinal axis. The circular-arc-shaped or ring-shaped guide groove connects ends of the plurality of engagement holes at a distal side. The peripheral groove extends in the circumferential direction in an outer peripheral surface. The plurality of vertical grooves are formed in the outer peripheral surface and extend from the proximal-end surface to the peripheral groove so as to connect the engagement holes and the peripheral groove. The guide groove, the peripheral groove, and the plurality of vertical grooves each have a width larger than a diameter of the injection needle, and the peripheral groove and the plurality of vertical grooves each have a width smaller than a diameter of the injector.

### REFERENCE SIGNS LIST

1 injection needle (injector)
2 endoscope sheath
3 syringe (injector)
4 cap
4a protrusion
5, 5′ sheath body
5A, 5A′ rotating section
5B stationary section
5B′ stationary section, endoscope injection positioning
device, body
5a guide groove
5b peripheral groove (outlet)
5c vertical groove
61, 62, 63 holder
61a, 62a, 63a, 61a′, 62a′, 63a′ engagement hole (holder)
7 first passage
8 second passage
8a inclined area
9 needle feed port (outlet)
10 feed port
11 drain port
12 liquid channel
100 injection kit

1. An endoscope sheath comprising:
an elongated sheath body having an elongated rotating section at a distal side thereof and a stationary section at a proximal side thereof, the rotating section and the stationary section being coupled to each other in a relatively rotatable manner about a longitudinal axis;
a first passage that extends through the sheath body along the longitudinal axis from a distal-end surface to a proximal-end surface thereof and into which an insertion section of an endoscope is insertable;
a second passage that is formed, in the sheath body, parallel to the first passage from the distal-end surface toward the proximal side and into which an injection needle is insertable;
an outlet through which a proximal end of the injection needle inserted in the second passage is pulled outside the sheath body, the outlet communicating with a proximal end of the second passage and provided at a radially outer side of the sheath body; and
a plurality of holders provided in the stationary section and separated by intervals in a circumferential direction around the longitudinal axis, the holders holding an injector having the injection needle pulled out through the outlet,
wherein each holder secures the injector, which is disposed at a protruding position where a distal end of the injection needle protrudes from a distal end of the sheath body via the second passage, to the stationary section in the circumferential direction and releases the injector at a released position located toward the proximal side relative to the protruding position, the released position being where the distal end of the injection needle is accommodated inside the sheath body, and
wherein, in a state where the injector is released from the holder, the outlet is capable of changing a pullout position of the proximal end of the injection needle relative to the stationary section in the circumferential direction as the rotating section and the stationary section are relatively rotated.

2. The endoscope sheath according to claim 1,
wherein a distal-end area of the second passage is inclined relative to the longitudinal axis gradually away from the longitudinal axis toward the distal end.

3. The endoscope sheath according to claim 1,
wherein the plurality of holders have a plurality of engagement holes provided in the stationary section and separated by intervals in the circumferential direction, each engagement hole being oriented toward the proximal side and being engageable with at least a part of the injector substantially in a longitudinal direction of the sheath body from the proximal side.

4. The endoscope sheath according to claim 3,
wherein each engagement hole holds the injector in a movable manner in the longitudinal direction between a maximum protruding position where further movement of the injector toward the distal side is limited and a recessed position located toward the proximal side relative to the maximum protruding position,
wherein the maximum protruding position is where the injection needle protrudes by a predetermined length from the distal end of the sheath body, and
wherein a moving distance of the injector between the maximum protruding position and the recessed position is larger than the predetermined length.

5. The endoscope sheath according to claim 3,
wherein the plurality of engagement holes are formed in a proximal-end surface of the stationary section and alternately connect with the second passage in the longitudinal direction in accordance with a relative angle between the rotating section and the stationary section around the longitudinal axis,
wherein the stationary section includes
a circular-arc-shaped or ring-shaped guide groove that connects ends of the plurality of engagement holes at the distal side,
a peripheral groove extending in the circumferential direction in an outer peripheral surface and serving as the outlet, and
a plurality of vertical grooves formed in the outer peripheral surface and extending from the proximal-end surface to the peripheral groove so as to connect the engagement holes and the peripheral groove, and
wherein the guide groove, the peripheral groove, and the plurality of vertical grooves each have a width larger than a diameter of the injection needle, and the peripheral groove and the plurality of vertical grooves each have a width smaller than a diameter of the injector.

6. An endoscope injection positioning device which is used together with an endoscope sheath having a first passage that extends therethrough along a longitudinal axis from a distal-end surface to a proximal-end surface thereof and into which an insertion section of an endoscope is insertable, and also having a second passage that is formed parallel to the first passage from the distal-end surface to the proximal-end surface and into which an injection needle is insertable, the endoscope injection positioning device comprising:
a substantially-cylindrical body having a passage into which the insertion section is insertable,
wherein the body includes
a plurality of engagement holes separated by intervals in a circumferential direction in the proximal-end surface, each engagement hole being engageable, in a central-axis direction from a proximal side, with at least a distal end of an injector connected to a proximal end of the injection needle inserted in the second passage, the plurality of engagement holes alternately connecting with the second passage in the central-axis direction in accordance with a relative angle with the endoscope sheath around the longitudinal axis,
circular-arc-shaped or ring-shaped guide groove that connects ends of the plurality of engagement holes at a distal side,
a peripheral groove extending in the circumferential direction in an outer peripheral surface, and
a plurality of vertical grooves formed in the outer peripheral surface and extending from the proximal-end surface to the peripheral groove so as to connect the engagement holes and the peripheral groove, and
wherein the guide groove, the peripheral groove, and the plurality of vertical grooves each have a width larger than a diameter of the injection needle, and the peripheral groove and the plurality of vertical grooves each have a width smaller than a diameter of the injector.
